(19) United States
(12) Patent Application Publication
(10) Pub. No.: US 2009/0198264 A1
(43) Pub. Date: Aug. 6, 2009

(54) Method and System for Improving Surgical Blades by the Application of Gas Cluster Ion Beam Technology and Improved Surgical Blades

(75) Inventors: Richard C. Svrluga, Newton, MA (US); Sean R. Kirkpatrick, Littleton, MA (US)

Correspondence Address: BURNS & LEVINSON, LLP, 125 SUMMER STREET, BOSTON, MA, 02110, US

(73) Assignee: EXOGENESIS CORPORATION, Wellesley Hills, MA (US)

(21) Appl. No.: 12/364,100

(22) Filed: Feb. 2, 2009

Related U.S. Application Data
(60) Provisional application No. 61/025,013, filed on Jan. 31, 2008.

(51) Int. Cl. A61B 17/32; B44C 1/22

(52) U.S. Cl. 606/167; 216/66

(57) ABSTRACT

Methods and systems for the improvement of a crystalline and/or poly-crystalline surgical blade include gas cluster ion beam irradiation of the blades in order to smooth; or to sharpen; or to reduce the brittleness and thus reduce susceptibility of the blade to crack, chip, or fracture; or to render the blades hydrophilic. Crystalline or poly-crystalline surgical blade (silicon for example) having a thin film cutting edge with improved properties.

### CROSS REFERENCE TO RELATED APPLICATIONS

This application claims priority from U.S. Provisional Patent Application Ser. No. 61/025,013, filed Jan. 31, 2008 and incorporated herein by reference in its entirety.

### FIELD OF THE INVENTION

This invention relates generally to cutting blades and knives such as surgical blades, and more particularly, to a method and system for improving the characteristics of crystalline and/or poly-crystalline surgical blades using gas cluster ion beam technology, and to improved surgical blades.

### BACKGROUND OF THE INVENTION

Recently surgical blades made of crystalline and/or poly-crystalline silicon have been introduced to the market for use in surgical cutting of mammal tissues for medical purposes. These blades offer several features that are advantageous over traditional metal blades and are economically advantageous over diamond blades. They can be manufactured relatively inexpensively and are often employed as single use disposable blades. While crystalline silicon has numerous advantages as a material for surgical blades, it also has at least one meaningful disadvantage. As a surgical blade material, silicon has the disadvantage of being brittle. Because of the brittle nature of silicon, especially crystalline silicon, the very sharp edge required for a surgical blade is susceptible to cracking and fracturing. This can result in spoiling of the cutting edge and/or the potential of shedding small pieces of material that may be left behind at the surgical site. This represents a significant problem, for example, when an ophthalmic surgeon uses such a blade and particles or small pieces of silicon are left behind in the ocular surgical site of a patient.

Gas cluster ions are formed from large numbers of weakly-bound atoms or molecules sharing common electrical charges and they can be accelerated to have high total energies. Gas cluster ions disintegrate upon impact and the total energy of the cluster ion is shared among the constituent atoms. Because of this energy sharing, the atoms are individually much less energetic than in the case of un-clustered conventional ions and, as a result, the atoms only penetrate to much shallower depths than would conventional ions. Surface effects can be orders of magnitude stronger than corresponding effects produced by conventional ions, thereby making important micro-scale surface modification effects possible that are not possible in any other way.

The concept of gas cluster ion beam (GCIB) processing has only emerged in recent decades. Using a GCIB for dry etching, cleaning, and smoothing of materials, as well as for film formation is known in the art and has been described, for example, by Deguchi, et al. in U.S. Pat. No. 5,814,194, “Substrate Surface Treatment Method”, 1998. Because ionized gas clusters containing on the order of thousands of gas atoms or molecules may be formed and accelerated to modest energies on the order of a few thousands of electron volts, individual atoms or molecules in the clusters may each only have an average energy on the order of a few electron volts. It is known from the teachings of Yamada in, for example, U.S. Pat. No. 5,459,326, that such individual atoms are not energetic enough to significantly penetrate a surface to cause the residual sub-surface damage typically associated with plasma polishing or conventional monomer ion beam processing. Nevertheless, the clusters themselves are sufficiently energetic (some thousands of electron volts) to effectively etch, smooth, or clean hard surfaces, or to perform other shallow surface modifications.

Because the energies of individual atoms within a gas cluster ion are very small, typically a few eV, the atoms penetrate through only a few atomic layers, at most, of a target surface during impact. This shallow penetration of the impacting atoms means all of the energy carried by an entire cluster ion is consequently dissipated in an extremely small volume in the top surface layer during an extremely short time interval. This is different from the case of ion implantation, which is normally done with conventional ions and where the intent is to penetrate into the material, sometimes penetrating several thousand angstroms, to produce changes in the surface and sub-surface properties of the material. Because of the high total energy of the cluster ion and extremely small interaction volume of each cluster, the deposited energy density at the impact site is far greater than in the case of bombardment by conventional ions and the extreme conditions permit material modifications including formation of shallow chemical conversion layers and forming shallow amorphized layers not otherwise achievable.

It is therefore an object of this invention to provide methods and apparatus for atomic-level surface smoothing of surgical blades for applications in mammalian medical surgery.

It is another object of this invention to provide methods and apparatus for surface modification of surgical blades for applications in mammalian medical surgery to reduce the susceptibility of the blade edges to cracking, chipping, and fracturing.

It is a further object of this invention to provide methods and apparatus for improving the sharpness of surgical blades for applications in mammalian medical surgery.

A still further object of this invention is to provide methods and apparatus for making the surface of a surgical blade for application in mammalian medical surgery more hydrophilic.

### SUMMARY OF THE INVENTION

The objects set forth above, as well as further and other objects and advantages of the present invention, are achieved as described hereinbelow.

One embodiment of the present invention provides a method of improving a silicon surgical blade having a cutting edge, comprising the steps of: disposing the blade in a reduced pressure chamber; forming a gas cluster ion beam in the reduced pressure chamber; irradiating one or more portions of the cutting edge of the blade with the gas cluster ion beam in the reduced pressure chamber to: smooth the one or more portions, sharpen the one or more portions, modify the chemical composition of the one or more portions, form compressive strain in the one or more portions, reduce the susceptibility to crack, chip, or fracture of the one or more portions or make the one or more portions hydrophilic.

The method may further comprise the steps of repositioning the blade within the reduced pressure chamber and irradiating one or more additional portions of the blade with the gas cluster ion beam in the reduced pressure chamber.

Another embodiment of the present invention provides a method of improving a silicon surgical blade having a cutting edge, comprising the steps of disposing the blade in a reduced pressure chamber, forming a gas cluster ion beam in the reduced pressure chamber, irradiating one or more portions of the cutting edge of the blade with the gas cluster ion beam in the reduced pressure chamber to: smooth the one or more portions; sharpen the one or more portions; modify the chemical composition of the one or more portions; form compressive strain in the one or more portions; reduce the susceptibility to crack, chip, or fracture of the one or more portions; or make the one or more portions hydrophilic.

The method may further comprise the steps of repositioning the blade within the reduced pressure chamber and irradiating one or more additional portions of the blade with the gas cluster ion beam in the reduced pressure chamber.

Yet another embodiment of the present invention provides a surgical blade made by any of the above methods. The blade may be silicon or substantially silicon. The blade may be a crystalline silicon blade.

Still another embodiment of the present invention provides a crystalline or poly-crystalline surgical blade having a thin film cutting edge. The crystalline or poly-crystalline blade may comprise silicon. The thin may be about 100 nm or less in thickness. The thin film may comprise SiO2, SiNX or SiCX. The thin film may be under compressive strain, have a hydrophilic surface, or be substantially amorphous.

For a better understanding of the present invention, together with other and further objects thereof, reference is made to the accompanying drawings and detailed description and in the appended claims.

### BRIEF DESCRIPTION OF THE DRAWINGS

FIGS. 1A through 1E show various possible configurations of prior art crystalline and/or poly-crystalline surgical blades;

FIG. 2 is a is a schematic view of a gas cluster ion beam processing system of the present invention;

FIG. 3 is an enlarged view of a portion of the gas cluster ion beam processing system showing the workpiece holder;

FIG. 4A is an enlarged schematic of a profile cross-section view of the cutting edge of a blade showing preferred geometry for GCIB irradiation for sharpening a first side of a cutting edge bevel according to an embodiment of the invention;

FIG. 4B is an enlarged schematic of a profile cross-section view of the cutting edge of a blade showing preferred geometry for GCIB irradiation for sharpening a two sides of a cutting edge bevel according to an embodiment of the invention;

FIG. 5 is an enlarged schematic showing a profile cross-section view of the cutting edge of a blade held in a fixture for GCIB irradiation according to an embodiment of the invention; and

FIG. 6A is an enlarged schematic of a profile cross-section view of the cutting edge of a blade showing GCIB irradiation of a side of a cutting edge bevel to improve the edge properties, according to an embodiment of the invention.

### DETAILED DESCRIPTION OF THE PREFERRED METHODS AND EMBODIMENTS

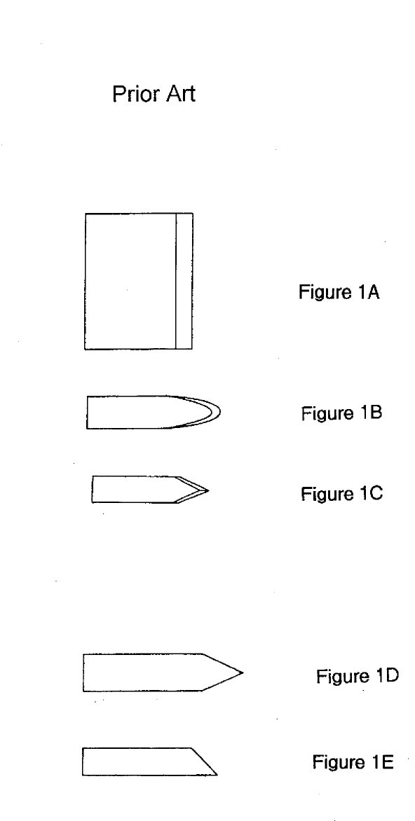

FIGS. 1A through 1E show a variety of prior-art configurations of crystalline and/or poly-crystalline blades. FIGS. 1A through 1C show plan views of exemplary blades to illustrate, in part, the wide range of blade configurations that can be constructed from crystalline and/or poly-crystalline materials using known techniques. FIGS. 1D and 1E, respectively, show side views of the cutting edges of such blades, which may be either dual-bevel as shown in FIG. 1D or single-bevel as shown in FIG. 1E. Different overall blade configurations as shown as examples in FIGS. 1A through 1C may be produced in either single- or dual-bevel configurations. It is clear that surgical blades may have multiple surfaces with different orientations—this factor somewhat complicates the concept of processing the surfaces with GCIB irradiation as required for the practice of the present invention.

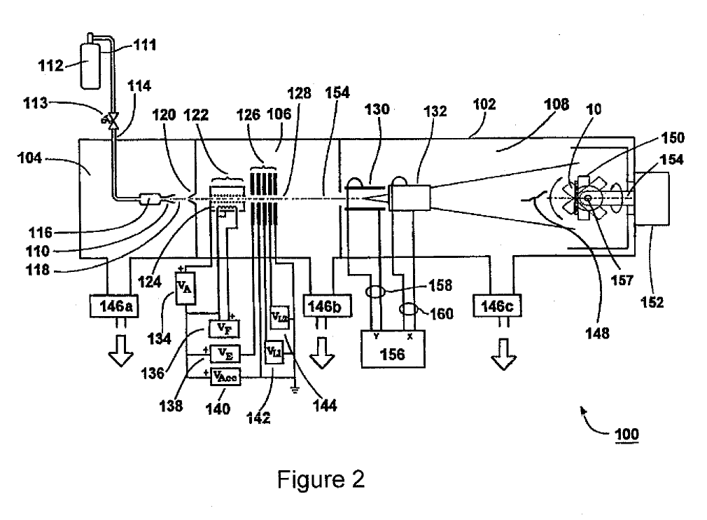

Reference is now made to FIG. 2 of the drawings, which shows an embodiment of the gas cluster ion beam (GCIB) processor 100 of this invention utilized for the surface modification of a surgical blade 10. Although not limited to the specific components described herein, the processor 100 is made up of a vacuum vessel 102 which is divided into three communicating chambers: a source chamber 104, an ionization/acceleration chamber 106, and a processing chamber 108 which includes therein a uniquely designed workpiece holder 150 capable of positioning the medical device for uniform smoothing by a gas cluster ion beam.

During the processing method of this invention, the three chambers are evacuated to suitable operating pressures by vacuum pumping systems 146a, 146b, and 146c, respectively. A condensable source gas 112 (for example argon, O₂, N₂, methane) stored in a cylinder 111 is admitted under pressure through gas metering valve 113 and gas feed tube 114 into stagnation chamber 116 and is ejected into the substantially lower-pressure vacuum through a properly shaped nozzle 110, resulting in a supersonic gas jet 118. Cooling, which results from the expansion in the jet, causes a portion of the gas jet 118 to condense into clusters, each consisting of from several to several thousand weakly bound atoms or molecules, and typically having a distribution having a most likely size of hundreds to thousands of atoms or molecules. A gas skimmer aperture 120 partially separates the gas molecules that have not condensed into a cluster jet from the cluster jet so as to minimize pressure in the downstream regions where such higher pressures would be detrimental (e.g., ionizer 122, high voltage electrodes 126, and process chamber 108). Suitable condensable source gases 112 include, but are not necessarily limited to argon or other noble gases, nitrogen, carbon dioxide, oxygen, nitrogen-containing gases, carbon containing gases, oxygen-containing gases, halogen-containing gases, and mixtures of these or other gases.

After the supersonic gas jet 118 containing gas clusters has been formed, the clusters are ionized in an ionizer 122. The ionizer 122 is typically an electron impact ionizer that produces thermoelectrons from one or more incandescent filaments 124 and accelerates and directs the electrons causing them to collide with the gas clusters in the gas jet 118, where the jet passes through the ionizer 122. The electron impact ejects electrons from the clusters, causing a portion the clusters to become positively ionized. A set of suitably biased high voltage electrodes 126 extracts the cluster ions from the ionizer 122, forming a beam, then accelerates the cluster ions to a desired energy (typically from 2 keV to as much as 100 keV) and focuses them to form a GCIB 128 having an initial trajectory 154. Filament power supply 136 provides voltage V_{F} to heat the ionizer filament 124. Anode power supply 134 provides voltage V_{A} to accelerate thermoelectrons emitted from filament 124 to cause them to bombard the cluster-containing gas jet 118 to produce ions. Extraction power supply 138 provides voltage V_{E} to bias a high voltage electrode to extract ions from the ionizing region of ionizer 122 and to form a GCIB 128. Accelerator power supply 140 provides voltage V_{Acc} to bias a high voltage electrode with respect to the ionizer 122 so as to result in a total GCIB acceleration potential equal to V_{Acc} volts. One or more lens power supplies (142 and 144, for example) may be provided to bias high voltage electrodes with potentials (V_{L1} and V_{L2} for example) to focus the GCIB 128.

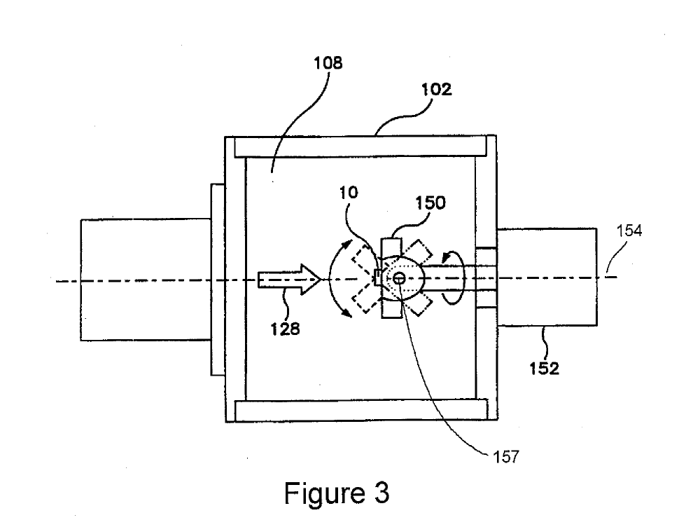

Referring now to FIG. 3, one or more surgical blades 10 to be processed by GCIB irradiation using the GCIB processor 100 is/are held on a workpiece holder 150, disposed in the path of the GCIB 128. In order to facilitate uniform processing of one or more surfaces or surface regions of the surgical blade(s) 10, the workpiece holder 150 is designed in a manner set forth below to position and/or manipulate the surgical blade 10 in a specific way.

As will be explained further hereinbelow, the practice of the present invention is facilitated by an ability to control the angle of GCIB incidence with respect to a surface of a surgical blade being processed. Since surgical blades may have multiple surfaces with different orientations, it is desirable that there be a capability for positioning and orientating the surgical blades with respect to the GCIB. This requires a fixture or workpiece holder 150 with the ability to be fully articulated in order to orient all desired surfaces of a surgical blade 10 to be modified, within the preferred angle of GCIB incidence for the desired surface modification effect. More specifically, when smoothing a surgical blade 10, the workpiece holder 150 is rotated and articulated by a mechanism 152 located at the end of the GCIB processor 100. The articulation/rotation mechanism 152 preferably permits 360 degrees of device rotation about longitudinal axis 154 and sufficient device articulation about an axis 157 that may be perpendicular to axis 154 to expose the surgical blade's cutting surfaces to the GCIB at angles of beam incidence from grazing angles of beam incidence to normal angles of beam incidence.

Referring again to FIG. 2: Under certain conditions, depending upon the size and the extent of the area of the surgical blade 10, which is to be processed, or when multiple blades are to be processed at the same time, a scanning system may be desirable to produce uniform irradiation of the blade or blades with the GCIB 128. Although not necessary for GCIB processing, two pairs of orthogonally oriented electrostatic scan plates 130 and 132 may be utilized to produce a raster or other beam scanning pattern over an extended processing area. When such beam scanning is performed, a scan generator 156 provides X-axis and Y-axis scanning signal voltages to the pairs of scan plates 130 and 132 through lead pairs 158 and 160 respectively. The scanning signal voltages may be triangular waves of different frequencies that cause the GCIB 128 to be converted into a scanned GCIB 148, which scans an entire surface or extended region of the surgical blade 10.

When beam scanning over an extended region is not desired, processing is generally confined to a region that is defined by the diameter of the beam. The diameter of the beam at the surgical blade's surface can be set by selecting the voltages (V_{L1} and/or V_{L2}) of one or more lens power supplies (142 and 144 shown for example) to provide the desired beam diameter at the workpiece.

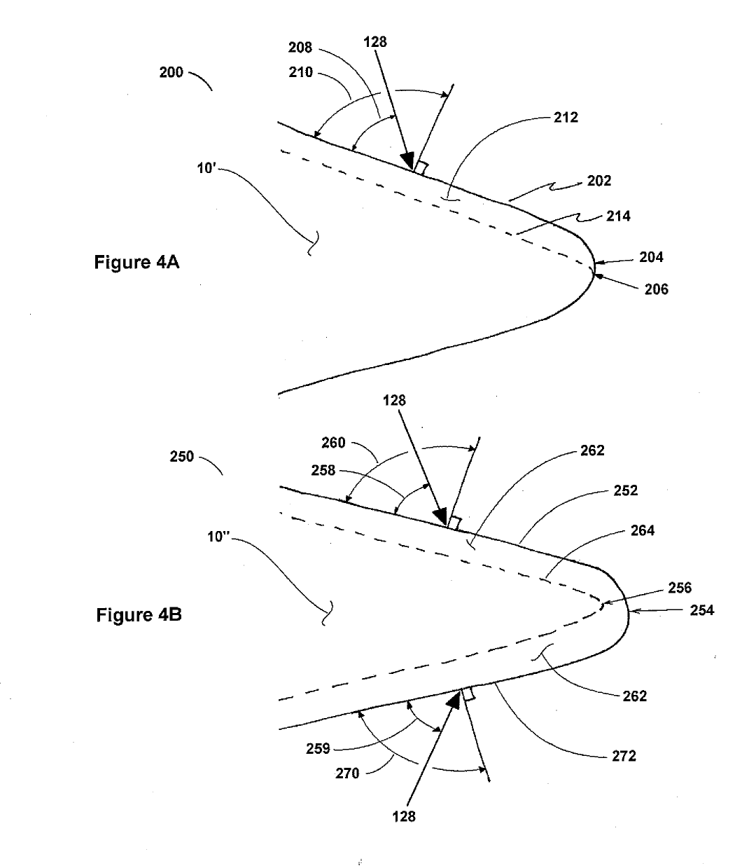

FIG. 4A is an enlarged schematic of a profile cross-section view of the cutting edge 200 of a surgical blade 10′ showing preferred geometry for GCIB irradiation for sharpening a first side of a cutting edge bevel according to an embodiment of the invention. The cutting edge has a surface 202 and an initial cutting edge radius 204 and is formed from a crystalline or poly-crystalline material such as, for example, silicon. The cutting edge is sharp and accordingly the cutting edge radius 204 is on the order of, for example, from about 5 to a few hundred nm. According to an embodiment of the invention, the cutting edge of the surgical blade 10′ is additionally sharpened by altering the shape of the blade by using a GCIB to etch away a portion 212 (not shown to scale) of a surface of a cutting edge bevel of the blade, resulting in a new cutting edge bevel 214 that results in a sharpened new cutting edge radius 206. For example, with an initial cutting edge radius 204 of 40 nm, upon removal of a portion 212, of from about 10 to 40 nm in thickness, there results a sharpened new cutting edge radius 206 of, for example 20 nm or less. For various initial cutting edge radii, and by selecting different depths of the portions 212 that are removed, it is possible to select desired different sharpening effects to achieve new cutting edge radii 206 of from a few nm to several tens or even hundreds of nm (but less than the initial cutting edge radius 204). For such processing, GCIB irradiation may be performed on a single cutting edge bevel surface (as shown in FIG. 4A), or on both cutting edge bevel surfaces forming the cutting edge (as shown in FIG. 4B). The thickness of the blade material (silicon in this exemplary case) that is removed from one or both cutting edge bevel surfaces, the portion 212, is typically less than 100 nm and is also typically less than or equal to the initial radius 204 of the cutting edge. Referring to FIG. 4A, the removal of portion 212 is done by GCIB etching of the cutting edge surface. A GCIB 128 is directed at the surface 202 of the cutting edge at an angle of incidence 208 falling in a range of angles 210 between grazing (0 degree) and normal (90 degree) incidence, with angles of incidence less than 90 degrees preferred because they tend to produce a greater sharpening effect. As described above, the GCIB may optionally be scanned over the cutting edge bevel surface to remove the portion 212 from as large an area of the bevel of the cutting edge as is desired.

Preferred gas cluster ion beams for etching crystalline or poly-crystalline blades are formed from (i) argon or other noble gases, or other inert gases, (ii) chemically reactive gases such as, for example, halogens or gases that are halogen compounds capable of etching silicon or other materials while forming volatile by-products, or (iii) chemically reactive gases such as, for example, O₂, N₂, or NH₃, which can form non-volatile compounds such as SiO₂ or SiN_{X} that may subsequently be removed by conventional chemical etching. The GCIB etching is performed using GCIB acceleration voltages within the range of about 2 kV to 100 kV, and with GCIB irradiation doses within the range of from about 10¹⁴ to about 10¹⁷ gas cluster ions per cm². Because the GCIB cluster ions disrupt upon impact with a surface, much of their kinetic energy becomes directed laterally to the direction of incidence on the surface. This results in a surface smoothing effect—thus the GCIB etching to sharpen the cutting edge also results in a smoothing effect on the cutting edge bevel, which has the effect of improving the cutting characteristics of the sharpened blade edge.

FIG. 4B is an enlarged schematic of a profile cross-section view of the cutting edge 250 of a surgical blade 10″ showing preferred geometry for GCIB irradiation for sharpening a both sides of a cutting edge bevel according to an embodiment of the invention. The cutting edge has a surface 252 and an initial cutting edge radius 254 and is formed from a crystalline or poly-crystalline material such as, for example, silicon. The cutting edge is sharp and accordingly the cutting edge radius 254 is on the order of, for example, from about 5 to a few hundred nm. According to an embodiment of the invention, the cutting edge of the surgical blade 10″ is additionally sharpened by altering the shape of the blade by using a GCIB to etch away a portion 262 (not shown to scale) of both surfaces of a cutting edge bevel of the blade, resulting in a new cutting edge bevel 264 that results in a sharpened new cutting edge radius 256. For example, with an initial cutting edge radius 254 of 40 nm, upon removal of a portion 262, of from about 10 to 40 nm in thickness, there results a sharpened new cutting edge radius 256 of, for example 10 nm or less. When both bevel edges are GCIB etched to remove the portion 262 for sharpening, it is preferable that the GCIB 128 irradiate the cutting edge twice, by first directing the GCIB 128 at the surface 252 of the cutting edge at an angle of incidence 258 falling in a range of angles 260 between grazing and normal incidence and by then directing the GCIB 128 at the surface 272 of the cutting edge at an angle of incidence 259 falling within a range of angles 270 between grazing and normal incidence, with angles of incidence less than 90 degrees preferred for both sides. As described previously, the GCIB may optionally be scanned over the cutting edge bevel surface to remove the portion 262 from as large an area of the bevel of the cutting edge as is desired.

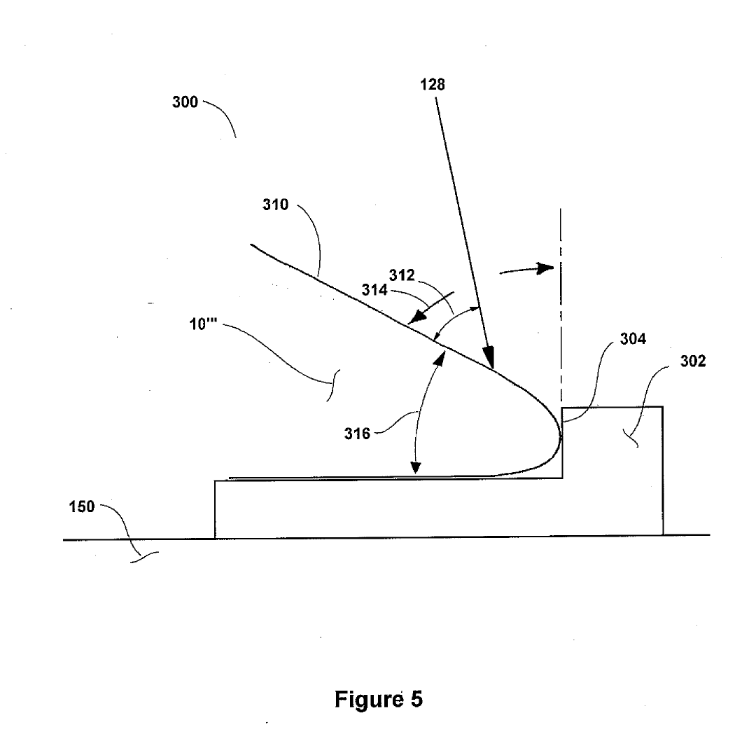

FIG. 5 is enlarged schematic showing a profile cross section view of the cutting edge 300 of a surgical blade 10′″ held in a fixture 302 attached to workpiece holder 150 for GCIB irradiation according to an embodiment of the invention. The fixture employs mechanical masking of the outmost edge of the cutting edge radius to optimize tip sharpness by preventing GCIB etching of the masked area. The surgical blade 10′″ has a bevel angle 316, and is held mounted against a masking edge 304 of the fixture 302. The masking edge 304 is shaped to follow the outline of the cutting edge of the blade so as to mask the cutting edge radius of all portions of the cutting edge. The surface 310 of one side of the bevel is irradiated by GCIB 128 at an angle of beam incidence 312 to the surface 310 in the range of angles 314 of from grazing incidence to (90 degrees less the bevel angle 316).

In addition to surgical blade sharpening and smoothing, in still other embodiments of the invention, GCIB irradiation is employed to improve the mechanical characteristics of a crystalline or poly-crystalline blade. The inherent brittleness of silicon cutting edges (and consequent tendency to crack, chip, and/or fracture) previously described is improved by GCIB modification. GCIB can be employed to change the physical and/or chemical composition of the silicon surface, resulting in a surface that is less susceptible to crack, chip, or fracture. By employing an inert GCIB, the silicon surface can be amorphized by destroying the crystallinity in a thin surface film and thus increasing its mechanical strength. Alternatively, by employing a chemically reactive GCIB, the chemical composition of a thin surface film on the cutting edge of the surgical blade can be modified. Such a modified surface film as for example a SiN_{X} film may be a material having a greater strength and durability than the original crystalline or poly-crystalline material. When a chemically reactive GCIB reacts to form a modified thin surface film and thereby incorporates additional material into a thin surface film by reaction incorporating non-volatile compounds, or when the film is amorphized, the film is placed under compressive strain, which reduces the likelihood of initiation of a crack or fracture in the film. The cutting surface of a silicon blade can also be made hydrophilic by GCIB treatment. Examples of change of chemical composition and of amorphization and of making the surface hydrophilic by using different source gases for the formation of the GCIB are shown in Table 1.

TABLE 1

| | | Exemplary | GCIB Acceleration Voltage |
|---|---|---|---|
| Case | Conversion | GCIB Source Gases | [GCIB Dose Range (ions/cm²)] |
| 1 | Si to SiO₂ | O₂, mixture of O₂ and noble gas | 2 kV to 100 kV |
| 2 | Si to SiNₓ | N₂, N-containing gas such as NH3, | [10¹⁴ to 10¹⁷] |
| | | mixtures of N-containing and noble gas | |
| 3 | Si to SiCₓ | C-containing gases such as CH₄ or | |
| | | C₂H₆, or mixtures of C-containing gases | |
| | | with noble gases | |
| 4 | Amorphization | Argon, other inert or noble gas, | 2 kV to 100 kV |
| 5 | Make Si surface | chemically reactive gases which create | [10¹³ to 10¹⁷] |
| | hydrophilic | non-volatile silicon compounds (but not | |
| | | halogen-containing or other etching) | |
| | | gases | |

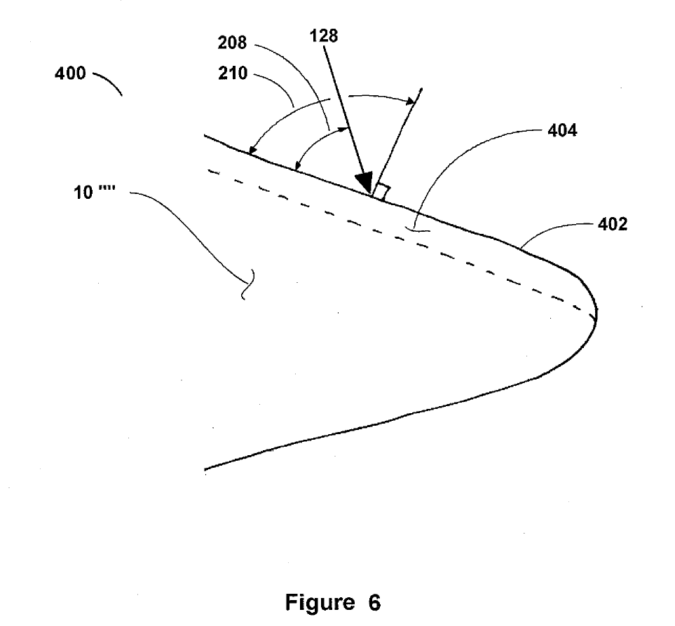

FIG. 6 is an enlarged schematic of a profile cross-section view of the cutting edge 400 of a surgical blade 10″″ showing preferred geometry for GCIB irradiation for surface modification of a first side of a cutting edge bevel of a crystalline or poly-crystalline blade according to embodiments of the invention for the processes tabulated in Table 1. The cutting edge has a surface 402 and is formed from a crystalline or poly-crystalline material such as, for example, silicon. A GCIB 128 having properties selected from Table 1, depending on the desired conversion, is directed at the surface 402 of the cutting edge at an angle of incidence 208 falling in a range of angles 210 between grazing and normal incidence, with angles of incidence less than 90 degrees preferred because they avoid a dulling effect on the cutting edge. The GCIB produces a converted film 404 (not shown to scale) at the irradiated surface 402. The thickness of the converted film 404 is dependent on the selected GCIB dose and acceleration voltage and may be selected in the range of from as little as about 10 nm to as much as about 100 nm. As described above, the GCIB may optionally be scanned over the cutting edge bevel surface to form the converted surface on as large an area of the bevel of the cutting edge as is desired. A fixture as shown previously in FIG. 5 may be used to mask the extreme edge of the cutting edge. When it is desired, both bevel surfaces of the cutting edge can be processed by repositioning the blade using the articulating workpiece holder 150 (FIG. 3) or by remounting the blade in the holding fixture 302 (FIG. 5).

Although the invention has been described with respect to various embodiments, it should be realized this invention is also capable of a wide variety of further and other embodiments within the spirit and scope of the appended claims.

What is claimed is:

1. A method of improving a crystalline or poly-crystalline surgical blade having a cutting edge, comprising the steps of:
disposing the blade in a reduced pressure chamber;
forming a gas cluster ion beam in the reduced pressure chamber;
irradiating one or more portions of the cutting edge of the blade with the gas cluster ion beam in the reduced pressure chamber to:
a) smooth the one or more portions;
b) sharpen the one or more portions;
c) modify the chemical composition of the one or more portions;
d) form compressive strain in the one or more portions;
e) reduce the susceptibility to crack, chip, or fracture of the one or more portions; or
f) make the one or more portions hydrophilic.

2. The method of claim 1, further comprising the steps of:
repositioning the blade within the reduced pressure chamber; and
irradiating one or more additional portions of the blade with the gas cluster ion beam in the reduced pressure chamber.

3. A surgical blade made by any of the methods of claim 1.

4. The blade of claim 3, wherein the blade is silicon or substantially silicon.

5. The blade of claim 3, wherein the blade is a crystalline silicon blade.

6. A method of improving a silicon surgical blade having a cutting edge, comprising the steps of:
disposing the blade in a reduced pressure chamber;
forming a gas cluster ion beam in the reduced pressure chamber;
irradiating one or more portions of the cutting edge of the blade with the gas cluster ion beam in the reduced pressure chamber to:
a) smooth the one or more portions;
b) sharpen the one or more portions;
c) modify the chemical composition of the one or more portions;
d) form compressive strain in the one or more portions;
e) reduce the susceptibility to crack, chip, or fracture of the one or more portions; or
f) make the one or more portions hydrophilic.

7. The method of claim 6, further comprising the steps of:
repositioning the blade within the reduced pressure chamber; and
irradiating one or more additional portions of the blade with the gas cluster ion beam in the reduced pressure chamber.

8. A crystalline or poly-crystalline surgical blade having a thin film cutting edge.

9. The blade of claim 8, wherein the crystalline or poly-crystalline blade comprises silicon.

10. The blade of claim 8, wherein the thin film is about 100 nm or less in thickness.

11. The blade of claim 8, wherein the thin film comprises SiO2, SiNX or SiCX.

12. The blade of claim 8, wherein the thin film is under compressive strain, has a hydrophilic surface, or is substantially amorphous.
